(19) United States
(12) Patent Application Publication
(10) Pub. No.: US 2016/0101174 A1
(43) Pub. Date: Apr. 14, 2016

(54) LENTIVIRAL VECTORS WITH TROPISM TO MOTOR NEURONS COMPRISING AN ANTIBODY THAT BINDS TO A PRE-SYNAPTIC TERMINAL RECEPTOR ON THE NEUROMUSCULAR JUNCTION AND A FUSOGENIC PROTEIN

(71) Applicant: IMPERIAL INNOVATIONS LIMITED, London (GB)

(72) Inventors: Nicholas D. MAZARAKIS, Richmond (GB); Ioanna ELEFTHERIADOU, London (GB); Antonio TRABALZA, London (GB)

(21) Appl. No.: 14/890,869

(22) PCT Filed: May 15, 2014
(86) PCT No.: PCT/GB2014/051490

§ 371 Date: Nov. 12, 2015

(30) Foreign Application Priority Data
May 15, 2013 (GB) 1308772.1

(51) Int. Cl. A61K 39/395; C07K 16/28; A61K 38/16; A61K 45/06; C07K 14/005

(52) U.S. Cl. CPC A61K 39/3955; A61K 45/06; C07K 14/005; A61K 38/162; C07K 16/2803; C07K 16/2878; C12N 2740/15043; C12N 2770/36132; A61K 48/00

(57) ABSTRACT

The present invention relates to vectors useful in the treatment of disease through targeted delivery. In particular, the present invention relates to vectors useful in the treatment of disease associated with neuronal degeneration, such as amyotrophic lateral sclerosis (ALS) and spinal muscular atrophy (SMA).

The present invention relates to vectors useful in the treatment of disease through targeted delivery. In particular, the present invention relates to vectors useful in the treatment of disease associated with neuronal degeneration, such as amyotrophic lateral sclerosis (ALS) and spinal muscular atrophy (SMA).

### BACKGROUND TO THE INVENTION

Delivery vectors (e.g. for gene therapy) developed from the lentiviruses, and in particular from Human Immunodeficiency Virus (HIV-1), have been shown to be efficient tools for the transduction of therapeutic agents such as transgenes into mammalian cells. Lentiviral vectors are produced by replacing the natural envelope protein with heterologous glycoprotein.

Among the first and still most widely used glycoproteins (GP) for pseudotyping lentiviral vectors is the GP derived from vesicular stomatitis virus (VSVG), which confers to various vectors the ability to deliver therapeutic agents (e.g. a gene) to a broad range of cell types and enables the production of stable high titre vector stocks, withstanding purification and concentration protocols required for their clinical use. Lentiviral vectors that efficiently transduce mammalian neurons have been successful at alleviating symptoms and extending survival in murine models of disease. Their broad cell tropism, however, do not make VSVG pseudotyped vectors amenable for targeting gene delivery to specific cell types (and hence disease sites) as they lack the ability to access difficult to reach areas (such as the central nervous system) without invasive delivery methods and in a manner that is atraumatic to the tissue.

Delivery of agents (e.g. transgenes) to the central nervous system (CNS) is particularly difficult because of the presence of the blood-brain barrier. Although the use of viral vectors has allowed sustained expression in neurons, vector administration into the CNS requires neurosurgery to deliver the vector to the target neuronal cell somata, by diffusion across a three-dimensional space usually in the range of cubic millimeters. This is clearly not applicable to devastating motor neuron disorders, such as amyotrophic lateral sclerosis (ALS) and spinal muscular atrophy (SMA), which have target cells located throughout the spinal cord.

Pseudotyping lentiviral vectors with specific viral glycoproteins can allow the targeting of these vectors to specific cell populations. For example, rabies virus glycoprotein (RVG) pseudotyping allows a non-invasive administration of the vector by targeting the peripheral sites of neuromuscular synapses in order to ultimately reach the affected central nervous system. Another pseudotype is based on adeno-associated virus (AAV2). A barrier to the clinical use of such pseudotyped lentivirus vectors, however, is the relatively low efficiency of transduction displayed by these vectors.

There is accordingly a need for minimally-invasive methods and vectors for administration of CNS-targeted therapeutics in diseases such as motor neuron (MN) diseases. There is a need for vectors and methods which can provide gene therapy to difficult to reach areas of the body, such as the central nervous system.

### SUMMARY OF THE INVENTION

The present invention addresses one or more of the above needs by providing a lentiviral vector comprising (i) an antibody that binds to a pre-synaptic terminal receptor on the neuromuscular junction (NMJ); and (ii) a fusogenic protein.

The antibody may be incorporated on the surface of the lentiviral vector. The antibody may be membrane-bound on the surface of the lentiviral vector.

The antibody may be an antibody that binds to a receptor on the NMJ which will endocytose (e.g. receptor-mediated endocytosis) the vector into (non-acidic) endosomes. The antibody may be an antibody against Thy-1 receptor, CAR (coxsackievirus and adenovirus receptor) and/or p75 (low-affinity neurotrophin receptor).

The use of other targeting moieties in place of an antibody is contemplated. Such targeting moieties include protein binding scaffolds. The term “antibody” includes antibody domain peptides (e.g. DABs), monoclonal antibodies, and antibody fragments such as Fab, F(ab)′2, Fv, ScFv, etc. Accordingly, the targeting moieties may be commercially available antibodies or binding scaffolds, which have been designed to achieve binding to the target cell or receptor in question. Alternatively, suitable antibodies and/or binding scaffolds can be prepared by conventional methods known to those skilled in the art.

An antibody may include at least one or two heavy (H) chain variable regions (abbreviated as VHC), and at least one or two light (L) chain variable regions (abbreviated as VLC). The VHC and VLC regions can be further subdivided into regions of hypervariability, termed “complementarity determining regions” (“CDRs”), interspersed with regions that are more conserved, termed “framework regions” (FRs). Preferably, each VHC 5 and VLC is composed of three CDRs and four FRs, arranged from aminoterminus to carboxy-terminus in the following order: FRI, CDRI, FR2, CDR2, FR3, CDR3, FR4. The VHC or VLC chain of an antibody can further include all or part of a heavy or light chain constant region.

The fusogenic molecule may be any glycoprotein, such as a viral glycoprotein, which will trigger endosomal release of the lentivirus capsid due to endosmal acidification. In one embodiment of the invention, the fusogenic molecule is mutated Sindbis glycoprotein. This mutated Sindbis glycoprotein is described in Lei, Y et al (2009) J Biol Eng, 3, 8; which is hereby incorporated by reference.

Other fusogenic molecules are described in WO 2006/130855, which is hereby incorporated by reference in its entirety. Thus, the fusogenic molecule may be a class I or class II fusogen. The fusogenic molecule may be pH sensitive. The fusogenic molecule may be hemagglutinin, which may be a mutant hemagglutinin. The fusogenic molecule may be SIN. The fusogenic molecule may consist of or comprise a viral glycoprotein derived from one of the following: Lassa fever virus, tick-borne encephalitis virus, dengue virus, hepatitis B virus, rabies virus, Semliki Forest virus, Ross River virus, Aura virus, Borna disease virus, Hantaan virus or SARS-CoV virus.

The vector of the invention may be a lentivirus pseudotyped with modified Sindbis GP incorporating a membrane bound antibody against presynaptic terminal receptors Thy1, p75NTR and/or CAR on the surface of the lentiviral vector.

By contrast with the lentiviral vectors described above, the lentiviral vector of the invention exhibits tropism to motor neurons (MNs) and furthermore preferentially transduces cells expressing such receptors. The present inventors have thus surprisingly found that the lentiviral vector of the invention achieves not only cell-specific targeting to the NMJ but also that targeting of a pre-synaptic terminal receptor on the NMJ permits retrograde axonal transport. It is believed that the specificity of the targeted vector is determined by the antibody incorporated on the surface of the vector. The present inventors have accordingly discovered that surface engineering of a lentiviral vector can confer both novel trafficking and specific transduction characteristics to the vector. This permits the restriction of transduction to specific cells and thus provides a safe and efficient means of targeted delivery and thus of in vivo gene therapy.

The lentiviral vector may accordingly further comprise a therapeutic agent which may vary according to the disorder to be treated. The therapeutic agent may be a CNS-targeted therapeutic agent. The therapeutic agent may be a neuroprotective protein, insulin-like growth factor or heat shock protein 70 (HSP-70). The therapeutic agent may be a neurotrophic factor.

The therapeutic agent may also be an agent for silencing dominant mutations (e.g. RNAi, siRNA). The therapeutic agent may be an agent for restoring protein function via gene replacement (e.g. by delivery of the wild-type gene). Thus, for treatment of spinal muscular atrophy type 1, the therapeutic agent may be the wild-type survival motor neuron 1 (SMN1) gene.

The lentiviral vector of the invention may be particularly useful for minimally invasive administration of the vectors by targeting the peripheral sites of the neuromuscular synapse in order ultimately to reach the affected CNS.

Accordingly the invention further provides a method of treating a neural disorder, the method comprising administering a lentiviral vector of the invention. In a related aspect, the invention provides the lentiviral vector of the invention for use in treatment of a neural disorder. In a further related aspect, the invention provides the use of the lentiviral vector of the invention in manufacture of a medicament for a neural disorder.

The neural disorder may be ALS. The neural disorder may be primary lateral sclerosis. The neural disorder may be progressive muscular atrophy. The neural disorder may be spinal muscular atrophy. The neural disorder may be progressive bulbar palsy. The neural disorder may be pseudobulbar palsy.

### BRIEF DESCRIPTION OF THE DRAWINGS

The present invention will now be described by way of example only with reference to the accompanying drawings, in which:

FIG. 1A shows a schematic drawing of plasmids used for production of targeted vectors. Targeted vectors were generated by 6 plasmid co-transfection using the 1) pαThy1.1/pαp75NTR/pαCAR plasmids and 2) Igαβ plasmids required for surface expression of antibody molecules 3) SINmu(SGN) envelope plasmid, the 4) 326-pRRLsincppt_CMV_eGFP-WPRE genome plasmid and the 289-pMD2-LgpRRE and 288-pRSV-Rev packaging plasmids;

FIG. 1B shows a schematic representation of the virus staining method for visualizing individual particles;

FIG. 1C shows αp75 NTR, α Thy1.1, αCAR, 5pI, and VSVG particles (overlaid upon coverslips) following staining for the presence of SGN fusogen (anti-HA-FITC), αp75 NTR/αThy1.1/αCAR (Alexa 594 anti-human IgG) and HIV p24 (anti-p24). The insert shows zoomed in view of the indicated region. Co-localizing pixels are indicated by a white signal. Scale bars, 10 μm;

FIG. 1D shows quantification of triple positive particles. For α75 49.8% of particles co-display antibody and fusogen molecule, while for αThy1.1 and for αCAR 31% and 53% of total particles are respectively triple positive;

FIG. 2A shows selective and efficient transduction in rat Schwan cells S-16 (p75NTR-192 positive), muscle L6 (p75 NTR-192 and Thy1.1 positive) and pheochromocytoma PC12 (p75 NTR-192 and Thy1.1 positive) which were spin infected with αp75 NTR and αTh1.1 at an MOI of 25. 5pI and αCD20 vectors were included as non-targeted control vectors to assess the specificity of targeting. 72 hrs post transduction, FACS analysis was conducted to analyze the percentage of EGFP expressing cells. Mean values are presented in the graphs: S-16, 57.93%±11.16 for αp75 NTR; PC-12, 3.8%±1.52% for αThy1.1, 4.91%±0.38% for αp75; L6, 23.25%±8.55% for αThy1.1, 8.20%±0.43% for αp75 (mean±s.d., n=3 experiments with different vector batches);

FIG. 2B shows selective and efficient transduction in human Hela and SH-SY5Y cells and mouse NSC-34 cells which were transduced with αCAR vector at MOI 25 and 50 respectively. 5pI vector was included as non-targeted control vector to assess the specificity of targeting. 72 hrs post transduction, FACS analysis was conducted to analyze the percentage of EGFP expressing cells. Mean values were: Hela, 8.00%±0.52% for αCAR; SH-SY5Y, 34.91%±4.79% for αCAR; NSC-34, 4.16%±1.59% (±s.d., n=3 experiments with different vector batches);

FIG. 3 shows targeting specificity of αp75 NTR, αThy1.1 and αCAR lentiviral vectors:

FIG. 3A shows the results of incubating 2.5×10⁵ L6, PC-12, and S-16 cells for 30 min at 37° C. with increasing concentrations of soluble antibodies anti-p75 NTR-192 (Abcam) and anti-Thy1.1 (Abcam) which recognize the same epitopes as αp75 NTR- and αThy1.1-targeted vectors respectively. Following antibody incubation, target cells were spin-infected with αp75 NTR- or αThy1.1-targeted vectors (MOI25). Cells were analysed for EGFP expression 72 h post transduction. In L6 and PC-12 cells (first row) the average transduction efficiency of targeted αThy1.1 was decreased by 45.6% and 63.3% respectively when spin-infection was performed in the presence of anti-Thy1.1 (9 μg/ml) blocking antibody. Transduction efficiency of αp75 NTR-targeted vector on S-16 and L6 (second row) was decreased by 62.5% and 33.8% respectively, in presence of soluble blocking anti-p75 NTR-MC192 (9 μg/ml). Transduction, in all cases, was slightly affected when soluble antibody with specificity different from that of the targeted vector was used. (n=3 experiments with different vector batches);

FIG. 3B shows the results of incubating 2.5×10⁵ SH-SY5Y cells for 30 min at 37° C. with increasing concentrations of soluble antibody anti-CAR-RmcB (Millipore) which recognize the same epitopes as αCAR lentiviral vector. Following antibody incubation, the cells were transduced with αCAR or α5pI vectors (MOI 25). Cells were analysed for EGFP expression 72 h post transduction. α5pI vector was used as non-targeted control. For αCAR (left column) transduction efficiency was decreased (60.22%) in presence of soluble blocking antibody anti-CAR-RmcB and only 9% in presence of isotype control. (n=3 experiments with different vector batches);

FIG. 4 shows gene transfer to primary motor neurons. EGFP expression in rat and mouse embryonic CNS primary motor neuron cultures infected at MOI 25 and 50 with αp75 NTR-192, αThy1.1, αCAR and 5pI control pseudotyped HIV-1 lentiviral vectors. Cultures were fixed 4 days after transduction and stained with antibodies to ChAT (Alexa594 secondary antibody staining motor neuron) shown in red, and GFAP (Alexa647 secondary antibody staining astrocytes) shown in blue. Graph shows the efficiency of gene transfer of pseudotyped vectors. Targeted vectors preferentially transduce MNs as compared to 5pI control which showed no specificity between MNs and astrocytes. The ratio EGFP+/ChAT+ relative to total ChAT+ cell number is plotted in black bars. The ratio of EGFP+/GFAP+ cell number relative to GFAP+ cell number is plotted on white bars. The values are expressed as mean±SD. *p<0.05, **p<0.01, ***p<0.0001. Scale bars, 50 μm. Each type of virus was assessed with the same culture batch in order to compare the differences in transduction levels. 5pI vector was used to control for non-targeted transduction. For each well 10 randomly chosen square fields with sides measuring 125 μm were counted with a computer-assisted imaging program (Rasband, W. S., ImageJ, U. S. National Institutes of Health, USA. http://imagej.nih.gov/ij/). Specific MN transduction was assessed as the percentage of double positive GFP/ChAT cells on the total of ChAT+ve cells. Specific astrocytic transduction efficiency was assessed as percentage of double positive EGFP/GFAP on the total of GFAP+ve cells;

FIG. 5 shows retrograde axonal transport of targeted vectors:

FIG. 5A shows primary motor neurons plated in microfluidic chambers incubated with either DiO labeled αp75 NTR or αThy1.1 or αCAR with TeNT-AF555 and anti-p75 NTR-AF647 for 2 hours at 37° C. and subsequently fixed and imaged by confocal microscopy. Shown are representative maximum projections of Z-stack images where white circles show examples of colocalisation of vectors with both markers and yellow circles show examples of colocalisation with just TeNT-AF555 and green circles show non-associated vectors. Scale bars, 20 μm;

FIG. 5B shows quantification of colocalisation (n=3, 286 particles αp75 NTR, 160 particles αThy1.1 and 210 particles αCAR);

FIG. 5C shows primary motor neurons incubated with either αp75, NTR αCAR or αThy1.1 DiO labeled vectors and anti-p75NTR-AF647 for 60 min and subjected to time-lapse confocal imaging of a region of axon>200 μm from the axonal compartment. Representative image series of trafficking vector particles showing DiO vectors and p75NTR (green and red in merged image respectively). Asterisks indicate examples of stationery particle. Kymographs of the same series. Scale bars, 10 μm;

FIG. 6 shows primary motor neurons incubated with EGFP expressing αp75 NTR or αThy1.1 or αCAR vectors or 5pI control for 6 h at 37° C. 72 h later, motor neurons were fixed, stained with antibodies against GFP, ChAT and SMI32 and imaged by confocal microscopy to assess transduction of soma. Cell bodies are located on the left; axons elongated through the microgrooves and reached the axonal compartment, located on the right. The analysis shown is representative of three independent primary motor neuron cultures. Scale bars, 100 μm;

FIG. 7 shows in vivo intramuscular delivery of αCAR-targeted vector: tropism of targeted lentiviral vector in the spinal cord following intramuscular delivery. Sections were double stained using anti-EGFP antibody (AlexaFluor 488 secondary antibody enhancing the GFP signal) shown in green, antibody to ChAT (AlexaFluor 594 secondary antibody staining MN) shown in red.

FIG. 8 shows in vivo intramuscular delivery of αCAR-targeted vector: in vivo bioluminescence imaging of mice intramuscularly injected with αCAR expressing luciferase. αCAR-targeted luciferase-expressing vector (25 μl Of 1.9×10⁹ TU/ml) and 5pI control (25 μl of 2.1×10⁹ TU/ml) was ulilateraly injected into TA of adult C57BL/6 mice (n=5 animals/group). Virus transduction at seven, fourteen and twenty-one days later, was determined by imaging the level of firefly luciferase reporter gene expression. In some cases stronger signal originating from the αCAR-targeted injected leg was masking photons emitted from spinal cord, and leg was covered to prevent camera saturation. This was not observed in animals injected with non-targeting vector (bottom row), supporting that the signal observed on the animals injected with the targeted vector originates from transduced cells at the level of spinal cord;

FIG. 9 shows in vivo intramuscular delivery of αCAR-targeted vector: tropism of targeted lentiviral vector in the spinal cord following intramuscular delivery. Sections were double stained using anti-EGFP antibody (AlexaFluor 488 secondary antibody enhancing the GFP signal) shown in green, antibody to ChAT (AlexaFluor 594 secondary antibody staining MN) shown in red. Insert show zoomed in optical sectioning images of a transduced MN. Scale bars: 250 μm for low magnification pictures, 50 μm for inserts.

### EXAMPLES

The work leading to this invention has received funding from the European Research Council Advanced Grant under the European Union's Seventh Framework Programme, ERC grant agreement number 23314.

### Methods and Materials

Hybridomas used in this study were:
(1) the OX-7 (Thy1.1) obtained from ECACC (84112008)
(2) the IgG192 (p75LNGFR) hybridoma cell line (kindly provided by Professor Giampietro Schiavo, London Research Institute) and
(3) the CAR (RmcB) hybridoma cell line obtained from ATCC (CRL-2379).

OX-7 cells were cultured in RPMI-1640 (Sigma, UK) supplemented with 2 mM L-glutamine and 10% Newborn Calf Serum (Heat Inactivated, Sigma, UK). 192 cells were cultured in RPMI-1640 supplemented with 10% Fetal calf serum, 1% penicillin/streptomycin, 2 mM L-glutamine and 1% sodium pyruvate. RmcB cells were cultured in RPMI-1640 (Gibco, UK), supplemented with 10% newborn Calf Serum, 2 mM L-glutamine, 10 mM Hepes and 1 mM sodium pyruvate. HEK 293T cells, obtained from ATCC, were maintained in a 5% CO2 environment in Dulbecco's modified Eagle's medium (Sigma, UK), with 10% Newborn Calf Serum (Heat Inactivated, Sigma UK), 1% Penicillin/Streptomycin and 1% L-glutamine (Sigma, UK). S-16 were obtained from ATCC (CRL-2941) and cultured in Dulbecco's Modified Eagle's Medium supplemented with 4 mM L-glutamine; PC-12 cells were obtained from ECACC (88022401) and cultured in RPMI 1640 medium supplemented with 2 mM L-glutamine, 10% Horse Serum and 5% Newborn Calf Serum in flasks which were pre-coated with 0.01% collagen type IV (Sigma C5533). L6.C11 were obtained from ECACC and cultured in Dulbecco's Modified Eagle's Medium supplemented with 10% Fetal calf serum, 1% penicillin/streptomycin, 2 mM L-glutamine. HeLa cells were obtained from ECACC (930210113) and cultured in EMEM (EBSS) supplemented with 2 mM L-glutamine, 1% Non-Essential Amino Acid (NEAA) and 15% Newborn Calf Serum. SH-SY5Y were obtained from ECACC (9430304) and cultured in Ham's F12: EMEM (EBSS) (1:1) supplemented with 2 mM glutamine, 1% NEAA and 10% Newborn Calf Serum. The mouse neuroblastoma cell line NSC-34 was obtained from Professor Pam Shaw (University of Sheffield) and was maintained in DMEM supplemented with 10% FBS, 1% essential amino acids (Sigma, UK) and 1% penicillin streptomycin and routinely passaged every 72-96 h.

For detection of individual viral particles, fresh viral supernatant from single plate preparation was overlaid upon Poly-D-Lysine (Sigma, UK) coated coverslips in 24-well culture plate, bound for 2 h at room temperature and fixed with 2% PFA for 15 min at room temperature. Coverlips were rinsed and with cold PBS twice and particles were immunostained for the presence of SINmu fusogen (anti-HA-FITC, 1:15, Miltenyi Biotech), αThy1.1/αp75NTR/αCAR human light chain (AlexaFluor® 594 anti-human IgG, 1:300, Invitrogen) and HIV p24 (1:300, Abcam, using an AlexaFluor® 633 secondary). Fluorescent images were acquired using a Leica SP5 II confocal microscope (Leica Microsystem, Gemany). Three independent images of each viral preparation, were used for quantification. On each image 5×10 μm fields were randomly selected, using custom macro in ImageJ (Rasband WS, ImageJ, U.S. National Institutes of Health, USA. http://imagej.nih.gov/ij/), and used to quantify/count the number of particles present. The number of positive particles in each of the three channels (SINmu(SGN), anti-IgG and p24) were counted manually. The percentage of triple positive particles was calculated.

Flow cytometry was performed to quantify transduction efficiency as follows: transduced EGFP expressing cells were detected with FL1 laser. Data where obtained with the (FACS machine) and analyzed with the FlowJo 8.7 software. Each FACS analysis was based on at least 10,000 events. Gates were set in a way that they contained less than 1% of the cells from the negative control cell population, which were untransduced cells for quantification of EGFP+ cells. To determine the titer of vector particles, cells transduced with serial dilutions of vector particles, as described above, were analysed for EGFP expression.

Immunocytochemistry of primary motor neuron (MN) cells was carried out as follows: MN cells were fixed with 2% paraformaldehyde (Sigma, UK) diluted in PEM/microtubules stabilization buffer (0.2 M PIPES, 10 mM MgCl2, 10 mM EGTA, pH 7.2) for 20 min in the dark, washed with PBS and permeabilised with PBS containing 2% BSA and 0.1% of Triton-X 100 (Sigma, UK) for 8-10 min. Blocking of nonspecific binding sites was achieved by 1 h incubation in PBS with 2% BSA. Antibodies to GFAP (1:800, MAB360, Millipore) and ChAT (1:100, AB144P, Millipore) were diluted in the same buffer and applied to the wells overnight at 4° C. Upon 30 min blocking, cells were incubated for 3 h at room temperature with secondary either donkey or goat anti-mouse and anti-rabbit antibodies, conjugated with Alexafluor®-594 or 647 (1:1000, Invitrogen, Paisley, UK). Following washes with PBS the wells' walls were removed from the slides and mounted using ProLong anti-fade aqueous mounting medium (Invitrogen, Paisley, UK).

Imaging of fixed motor neurons was carried out as follows: cells were treated as described in the figure legends above. Following treatments neurones were fixed in 4% PFA, and then quenched in PBS with 100 mM glycine, before permeabilisation in PBS plus 2% BSA and 0.1% Triton-X 100, and addition of primary antibodies. Staining was visualised using AlexaFluor® secondary antibodies (Invitrogen, Paisley, UK) as described in figure legends. Cells were imaged using a Leica SP5 II confocal microscope and processed using ImageJ and images enhanced using a 1.0 pixel median filter and brightness/contrast and were then rendered and pseudocoloured using ImageJ.

All animal procedures were approved by the local Ethical Committee and performed in accordance with United Kingdom Animals Scientific Procedures Act (1986) and associated guidelines. All efforts were made to minimize the number of animals used and the suffering. Animals were supplied by Charles River, UK and housed under a 12 hour light/dark cycle with food and water available ad libitum.

### Example 1

### Production of Lentiviral Vectors

Lentiviral vectors with tropism to motor neurons (MNs) via the neuromuscular junction (NMJ) were created as follows.

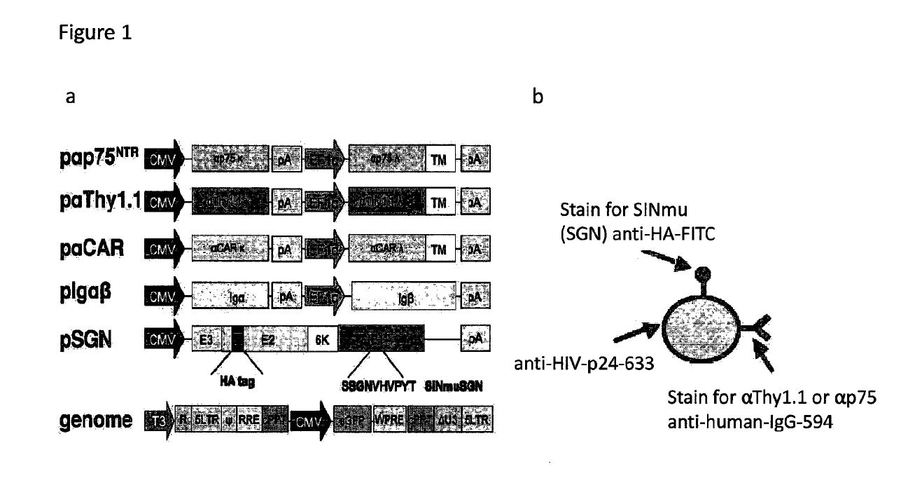

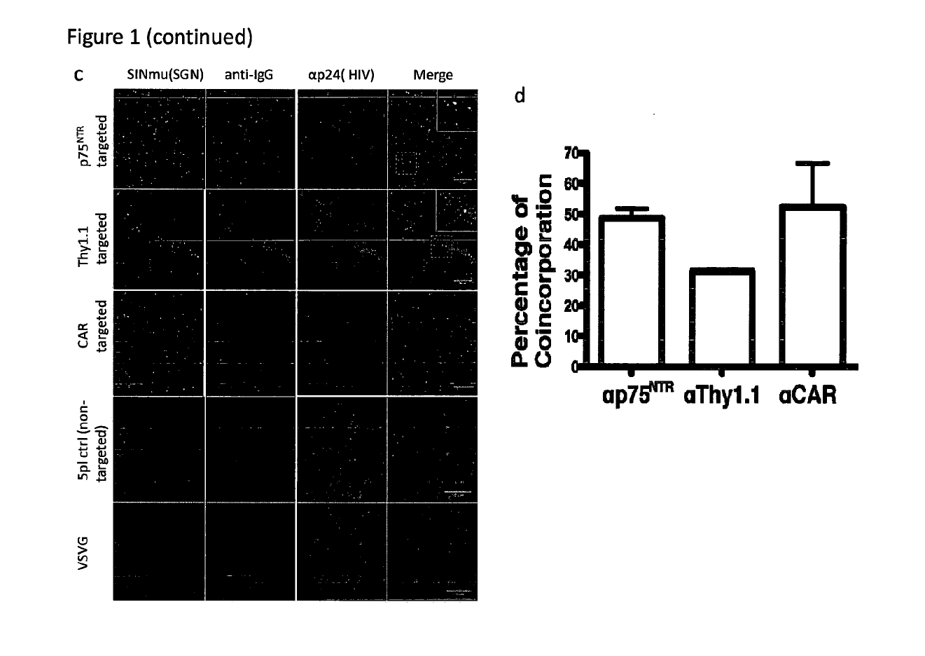

The variable regions of heavy and light chain of mouse antibodies specific for rat p75NTR, rat Thy1.1 (CD90) and CAR (RmcB) presynaptic terminal receptors were amplified and cloned upstream of the human constant regions in pαCD20 to create pαThy1.1 (CD90)-, pαp75NTR- and pαCAR-targeted vectors (FIG. 1A).

The expression plasmids pαThy1.1, pαp75NTR and pαCAR, encoding for the chimeric membrane bound antibodies for targeting were generated by amplification of heavy and light chain variable regions of murine anti-Thy1.1, anti-p75NTR (clone MC-192) and anti-CAR (clone RmcB) antibodies. RNA was extracted from using the RNeasy Mini Kit (QIAGEN, UK). First strand cDNA (RACE-ready cDNA) synthesis was performed using a modified lock-docking oligo(dT) anti-sense primer and the SMARTer II A oligo (SMARTer RACE cDNA Amplification Kit; Clontech). Heavy and light chain variable regions were PCR amplified (5′RACE). First round 5′ RACE was performed using UPM (universal Primer mix; Clontech) and constant region degenerate Hvbw(deg) and Lvbw(deg) primers. 5′ RACE PCR reactions were performed using the Advantage 2 Polymerase Mix (Clontech) and RACE PCR products were cloned to pCR 2.1-TOPO (Invitrogen, UK) and sequenced. Second round PCR amplification was performed with gene specific primers using the Platinum Pfx DNA Polymerase (Invitrogen, UK), and blunt-end PCR products were cloned to pCR-Blunt II-Topo (Invitrogen, UK) and sequenced. Primers sequences used for amplification of heavy and light chains were designed to insert the coding sequences for XhoI and NheI restriction enzymes (heavy chain) and HindIII and BsiWI restriction sites (light chain) that will facilitate cloning. Final PCR products were cloned upstream human heavy and light chain constant regions of the pαCD20 plasmid. The resulting constructs were designated as pαThy1.1, pαp75 and pαCAR. Constructs pαCD20, pIgαβ, and pSINmu(SGN) were kindly provided by Dr P. Wang (University of Southern California, Los Angeles, Calif. 90089).

Production of recombinant non-replicative HIV-1-based lentiviral vectors was performed by a six-plasmid co-transfection of HEK293T cells as described in Yang, L et al (2006) Proc Natl Acad Sci USA, 103, 11479-84; and Lei, Y et al (2009) J Biol Eng, 3, 8; both of which are incorporated herein by reference thereto. Briefly, 293T cells (12×150 mm dishes; 1.4×107 cells per dish) at 80% confluence, were transfected with 5 μg vector plasmid (pRRLsincppt-CMV-eGFPWPRE kindly provided together with HIV packaging plasmids by Professor James Uney, University of Bristol, Bristol, UK), 2.5 μg of each of the packaging vector plasmids expressing the HIV-1 gag/pol gene (pMD2-LgpRRE), HIV-1 Rev (pRSV-Rev) together with 2.5 μg of each of pαIgαβ (encodes human Igα and Igβ) two immunoglobulin associated proteins that are required for surface expression of antibodies), pSINmu and either pαThy1.1, pαp75 or pαCAR (encoding mouse/human chimeric anti-Thy1.1, anti-p75 and anti-CAR antibodies respectively), following the addition of 1 M CaCl2. 16 h post transfection medium was replaced with fresh low serum (2%) media supplemented with 10 mM sodium butyrate. 72 hours post transfection, the viral supernatant was collected and filtered through a 45-μm pore size filter. VSVG, 5pI and αCD20 pseudotyped vectors were used throughout as controls.

Large-scale preparations were concentrated by centrifugation at 6000 r.p.m. for 12-16 h at 4° C. (Beckman Coulter Avanti J-E, F500 rotor, Beckman Coulter, High Wycombe, UK). The pellet was resuspended in ice-cold phosphate-buffered saline (PBS), and was further concentrated by ultracentrifugation for 90 min at 20,000 r.p.m., 10° C. (Beckman Coulter Optima L-80XP). The pellet was resuspended in 50 μl ice-cold TSSM formulation buffer (20 mM Tromethamine, 100 mM NaCl, 10 mg/ml sucrose and 10 mg/ml mannitol) (2000-fold concentration).

Independently, a lentiviral vector bearing an isotype control antibody (here αCD20) and a lentiviral vector lacking the membrane bound antibody (5pI control) were produced and used as negative targeted and non-targeted controls, respectively.

Three versions of targeted vectors were produced encoding either enhanced GFP (EGFP), LacZ or Luciferase (Firefly) reporter genes all driven by human Cytomegalovirus promoter (CMV).

To determine the percentage of lentiviral particles that had incorporated both anti-Thy1.1 or anti-p75NTR or anti-CAR (antibody) and fusogenic protein (SINmu) on their surface, LacZ expressing HIV-1-virions were produced and subsequently immunofluorescently stained by a triple labeling method (FIG. 1B). As controls the non-targeted 5pI vector (lacking the surface antibody required for targeting) were included as well as virions pseudotyped with the Vesicular Stomatitis Viral Glycoprotein (VSVG) (non-IgG pseudotyped control) lacking both the antibody and the fusogenic protein. Confocal microscopy imaging and subsequent quantitation revealed that particles positive for HIV-p24, surface antibodies and SGN fusogen were present in the αp75NTR, αThy1.1 and αCAR samples (FIG. 10). For αp75NTR-CMV-SGN 49.8% of particles were triple positive, while for αThy1.1-CMV-SGN 31% and for αCAR-CMV-SGN 53% of particles co-displayed the desired features. In all cases the SGN/IgG only positive structures, were excluded from counting (FIG. 1D). As expected, colocalisation of 5pI control only with the SINmu was observed, whereas no colocalization of the virions with either protein (antibody or fusogenic protein) was detected for VSVG (FIG. 1D).

For screening of the fusogenic molecule (SINmu) and the membrane bound antibody (human-IgG) by western blot analysis, for single particle imaging analysis and for fixed imaging and live imaging trafficking studies, lentiviral vector particles were generated by single-plate 6 plasmid co-transfection. Transfections were performed as described above. Thirty-six hours post induction viral supernatant from each plate was collected, filtered through a 0.45 μm pore size and the viral supernatant was transferred into a sterile polypropylene ultra-centrifugation tube. A sucrose cushion was created by underlying to the bottom of the tube 2 ml of 20% sucrose solution. The preparations were purified/concentrated by ultracentrifugation for 2 h at 81,949 g at 4° C. (Beckman Coulter Optima L-80XP) and the pellet was left to dissolve overnight at 4° C. in 60 μl ice-cold TSSM formulation buffer and next day aliquoted into single-use cryovials and stored at −80° C.

To label the particles with lipophilic dye (Vybrant DiO, Invitrogen, UK), an additional step was added. 16 h post-transfection media was replaced with Opti-Mem (Invitrogen, UK) containing 3.7 mM Vybrant dye and incubated at 37 degrees C. for 2 h before replacing with media containing sodium butyrate as above.

Although production of lentiviral vectors involving 6 plasmid co-transfection might be thought to be challenging, the biological titers achieved were high (within the range of ×10¹⁰ TU/ml) and comparable to 4 plasmid co-transfection VSVG controls (biological titer 10¹⁰-10¹¹ TU/ml). Even with a small percentage of potent active lentiviruses (40-50% of the particles produced co-display the two key molecules required for targeted cell transduction) we report efficient in vitro targeted transduction. Data generated in our lab showed that by altering the signal peptide sequence of the antibodies used for targeting, to match the origin of the producer cell line we could increase the incorporation levels by 15-20% (data not shown). This opens the possibility to further improve the system in future studies.

### Example 2

### The Lentiviral Vectors Exhibit In Vitro Transduction of Cells

To evaluate the targeting potential of these vectors rat, mouse and human cell lines expressing the targeted receptors were incubated with target vectors and controls (MOI 25). Rat S-16, L6 and PC-12 cells (2.5×10⁵) were plated in a 24-well culture dish and spin infected with concentrated targeted vectors (αp75, αThy1.1) at MOI 25 as described in Yang, L et al (2006) Proc Natl Acad Sci USA, 103, 11479-84; incorporated herein by reference. 5pI and αCD20 vectors were included as non-targeted control vectors to assess the specificity of targeting. FACS analysis was performed to determine the percentage of EGFP expressing cells.

Human HeLa, SH-SY5Y and mouse NSC-34 cells (1.5×10⁵) were plated in a 24-well culture dish and infected with concentrated targeted vector αCAR and 5pI control for 6 hours at 37° C. with 5% CO₂.

EGFP-expressing lentiviral vectors allowed quantitative assessment of transduction in vitro as monitored by flow cytometry (Becton Dickinson LSR Benchtop Flow Cytometer, Becton Dickinson, San Jose, Calif., USA). Briefly, HEK 293T cells were seeded in 12-well plates at 5×10⁵ cells per well. Eighteen hours upon seeding cells were transduced for 6 h with serial dilutions of the vector being quantified, in the presence of 8 μg/ml polybrene. Seventy-two h post transduction the percentage of EGFP-positive cells was determined by flow cytometry. The viral titer was calculated using the following formula: Transduction units (TU/ml)=(percentage of fluorescent positive cells/100)×(number of cells per well on day of transdcution)×(vector dilution factor) per μl of vector. For vectors expressing the LacZ gene, HEK293T cells were plated in a 12 well plate (300,000 cells/well). 24 hours later cells were transduced with vector (plus 8 □g/ml polybrene). 48 h post transduction cells were washed and fixed in PFA and LacZ expression detected using the □-Gal Staining kit (Invitrogen, K1465-01). Positive cells were then manually counted to determine number of transducing units per ml.

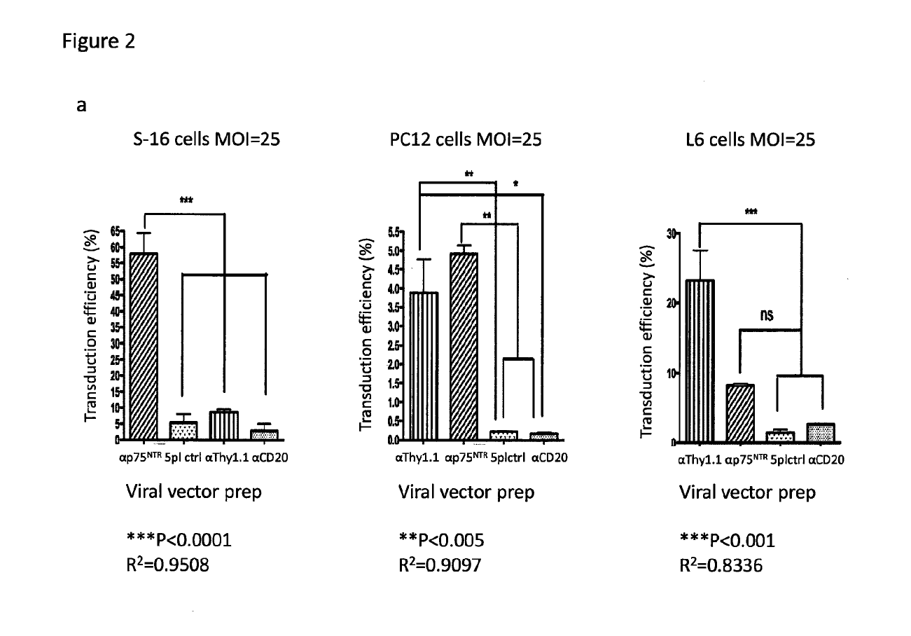

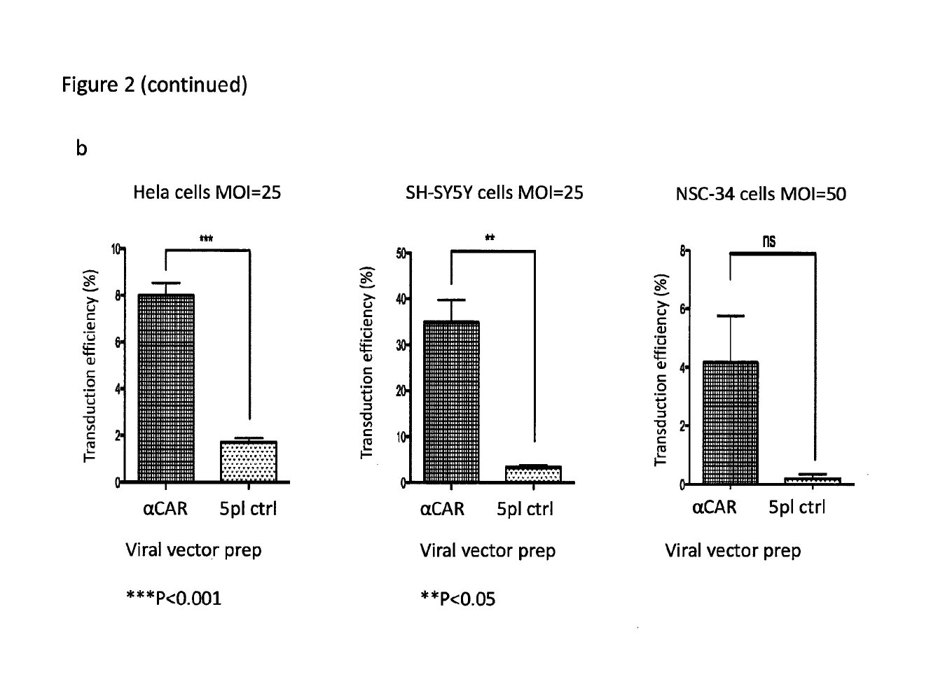

Rat S-16 Schwann cells were used as targeted cell line for αp75NTR targeted vector (expressing p75NTR but not Thy1.1 or CD20 receptors). When S-16 cells were infected with αp75NTR vector and αThy1.1, αCD20 and 5pI control vectors, efficient transduction was observed only for αp75NTR-targeted vector. Transduction levels observed for αp75NTR vector were as high as 57.93%±11.16% (mean±s.d.; n=3 experiments with different vector batches), whereas transduction observed from αThy1.1-, αCD20-targeted and 5pI non-targeted control vectors was consistently below 5% and thus considered as background (FIG. 2A). Rat pheochromocytoma PC-12 and muscle L6 cells, both expressing only p75NTR and Thy1.1 target receptors, were transduced with αp75NTR- and αThy1.1-targeted vectors and controls (αCD20 and 5pI). Whereas the αCD20-targeted and 5pI non-targeted control vectors resulted in low-level background transduction (below 0.5% and 2% respectively), the αThy1.1- and αp75NTR-targeted vectors efficiently transduced the target cell (FIG. 2A). αThy1.1-targeted vector transduced PC-12 cells at low levels (3.8%±1.52%; mean±s.d., n=3 experiments with different vector batches), while higher transduction efficiency was consistently reported for L6 cells (23.25%±8.55%; mean±s.d., n=3 experiments). Similarly αp75NTR vector transduced both PC-12 and L6 cells at different transduction levels (4.91%±0.38% for PC-12 and 8.20%±0.43% for L6). Consistently, we did not detect off-target transduction when we transduced target cell lines with αCD20 and 5pI vectors.

When αCAR targeted vector was applied to its target cells human HeLa and SH-SY5Y and mouse NSC-34 (MOI 25) it transduced all cell lines. Efficiency of transduction varied between different cell lines for the targeted vector, though transduction resulting from non-targeted 5pI control was at background levels (FIG. 2B). Cells were transduced at MOI 25 (data not shown) and MOI 50. Interestingly, when the MOI was increased from 25 to 50 in order to achieve significant levels of transduction with the αCAR-targeted vector, transduction observed from 5pI control remained below background levels (FIG. 2B).

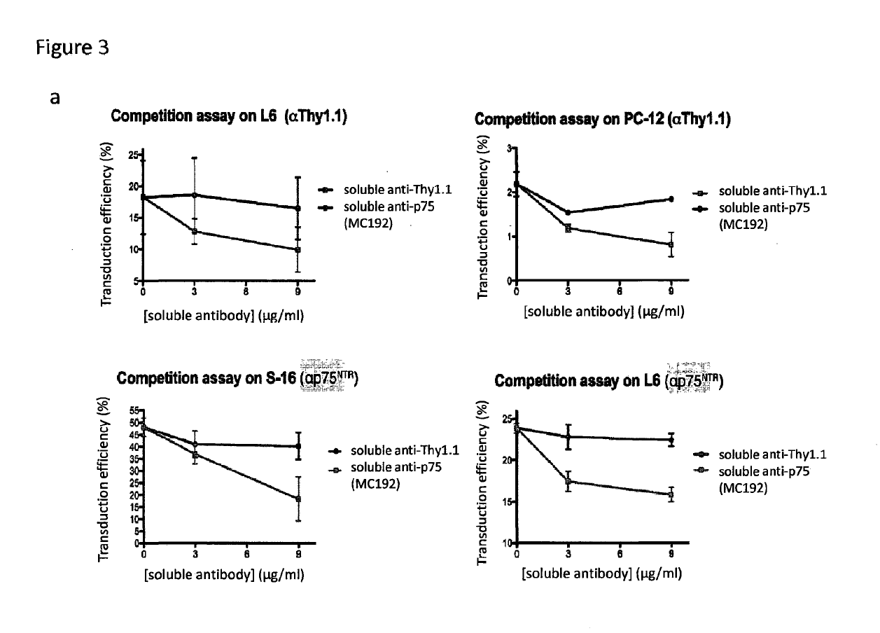

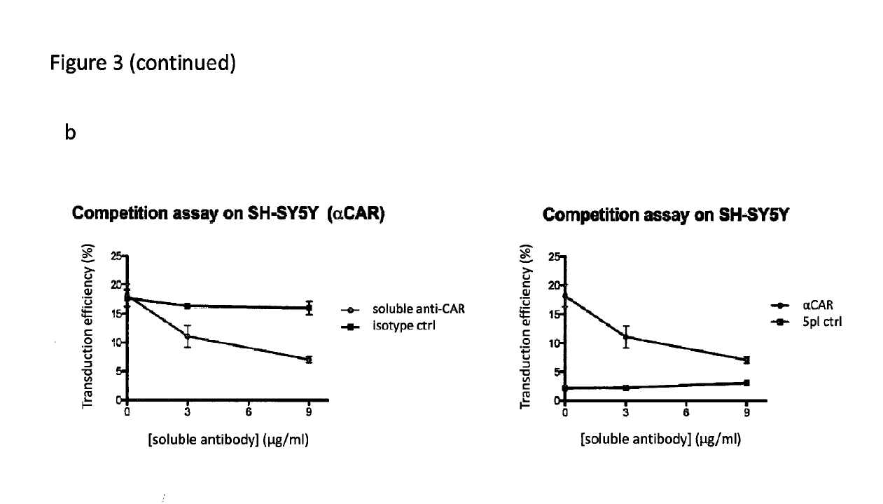

To determine whether the specificity of transduction observed was a consequence of antibody-target receptor interaction a series of competition assays was performed. Target cells (rat S-16, L6 and PC-12; human SH-SY5Y) (2.5×10⁵) were infected with targeted vectors for 6 h in presence of increasing concentrations of soluble anti-p75-192 (Abcam), anti-Thy1.1 (Abcam) and anti-CAR (RmcB) (Millipore) antibodies. FACS analysis was then performed to quantify transduction efficiency. Gene transfer to target cell lines, mediated by targeted vectors could be blocked with these antibodies (FIG. 3).

FIG. 3 presents the effect of blocking antibody and isotype control in the transduction achieved by the αp75NTR, αThy1.1 and αCAR targeted vectors (n=3). Error bars are indicative of the variability observed between individual experiments.

For all three αp75NTR-, αThy1.1- and αCAR-targeted vectors, the level of EGFP+ cells was reduced in the presence of soluble antibody specific for p75NTR, αThy1.1 and αCAR. The specificity was further confirmed by including in the competition assay the non-targeted 5 plasmid control vector for which transduction efficiency remained unaffected at background levels (FIG. 3B right).

### Example 3

### The Lentiviral Vectors Exhibit Targeted Transduction of Primary Motor Neurons

The transduction efficiency of targeted vectors in embryonic primary MN cultures was examined. Rat and mouse primary cell cultures of MNs (1×10⁵ cells per well) were plated in eight-well chambered slides. Transduction of rat primary neurons was carried at DIV2 with concentrated targeted and 5pI control vector preparations (MOI25 and 50). Transduction experiments were carried out in triplicate for each MOI for all LV preparations. Primary neuronal cultures were incubated with vector stocks in 300 ml conditioned culture medium for 6 h at 37° C. with 5% CO2. After 6 h, medium was replaced with fresh conditioned cultured medium and cells were incubated for a further 3 days at 37° C. with 5% CO₂.

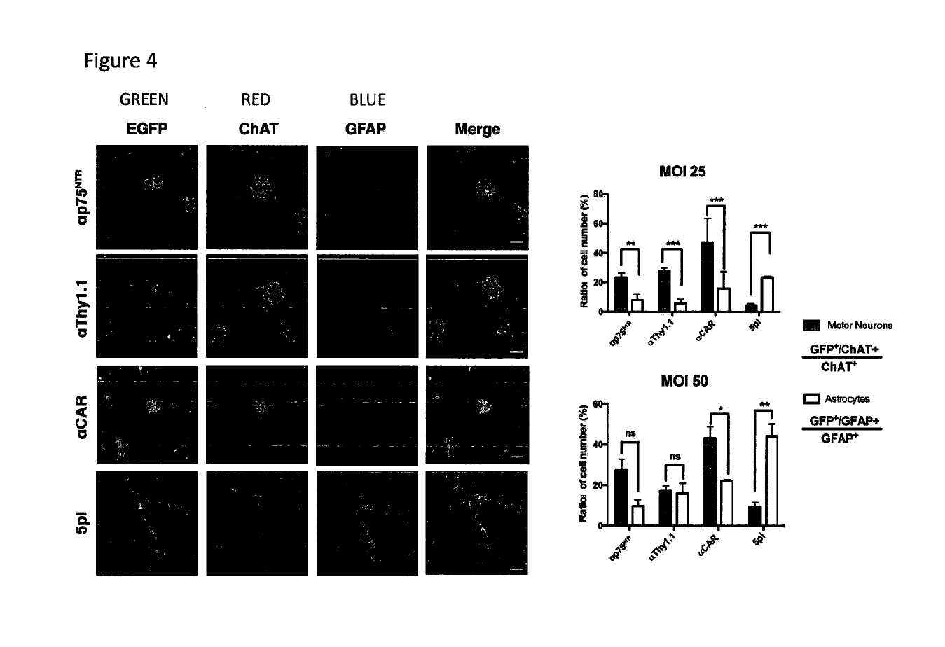

These cultures are enriched in neurons (about 80% at first day in vitro, which was the time of the transduction) but also contain glia (GFAP+ astrocytes), which increase with time in culture (data not shown). The expression of the targeted receptors (rat Thy1.1 and p75NTR and mouse CAR) in rat and mouse primary motor neurons in culture has been assessed and verified by immunohistochemistry prior to proceeding with transduction experiments (data not shown). Each type of virus was assessed with the same culture batch in order to compare the differences in transduction levels. Targeted lentiviral vectors preferentially transduced motor neuron cells compared to non-targeted control, which was shown to have no specificity between MNs and astrocytes (FIG. 4). To quantitatively assess the three targeted vectors the motor neuron marker ChAT was used, which revealed that 70.8±1.5% of the EGFP+ cells transduced with αp75NTR, 65.3±2% with αThy1.1 and 66±1.7% with αCAR targeted vector (mean±s.d.; in all cases n=3 experiments) were indeed motor neurons, whereas only 12.52±3.62% of the cells transduced with 5pI control were ChAT+. Glial fibrillary acidic protein (GFAP) immunolabeling defined the percentage of astrocytes transduced by targeted and non-targeted vectors.

More specifically, at an MOI 25, p75NTR-targeted vector was found to transduce 23.15±8.51% of ChAT+ cells and 8.2±2.7% of GFAP+ cells, whereas at an MOI of 50 it transduced 27.34±9% of ChAT+ cells and 9.8±4.48% of GFAP+ cells. Thy1.1-targeted vector transduced, at MOI 25, 27.85±3.75% of ChAT+ cells and 5.64±3.81% of GFAP+ cells, whereas at MOI 50, this vector transduced 17.15±3.31% of ChAT+ cells and 15.8±9.7% of GFAP+ cells. Finally αCAR-targeted vector, at MOI 25, transduced 47.08±25% of ChAT+ cells and 15±9% of GFAP+ cells, whereas at MOI 50, it transduced 43.2±10.8% of ChAT+ cells and 18±4.7% of GFAP+ cells. The non-targeted control at MOI 25, transduced 4.38±1.27% of ChAT+ cells and 23.17±7.7% of GFAP+ cells, whereas at MOI 50, it transduced 9.52±3.48% of ChAT+ cells and 44.18±7.8% of GFAP+ cells. Although an increase in the transduction levels of the motor neurons was observed when increasing the MOI (from 25 to 50), the percentage of astrocytes being transduced inevitably increased. At MOI 50 there was no significant difference in preference of the population being transduced by the targeted vectors, although the 5pI control still preferentially transduced astrocytes.

Interestingly, extensive cell death was observed upon application of the αp75NTR-targeted lentiviral vector to the primary motor neurons. Addition of αp75NTR-targeted vector resulted in axonal retraction and eventually loss within the first 2.5 hours of the incubation (data not shown). To avoid this the incubation time was limited only for αp75NTR. This observation further proved the specificity of this targeted vector, since it has been shown that activation of p75NTR in embryonic stages activates apoptosis (19).

### Example 4

### The Lentiviral Vectors Exhibit Retrograde Trafficking in Primary MNs

It is possible that the endocytic trafficking itinerary varies depending upon which part of the axon the vector internalizes (e.g. only axon tips allow retrograde transport). To investigate if this was the case and to unambiguously demonstrate that p75NTR-, Thy1.1- and CAR-targeted vectors enter the motor neurons via axon tip and retrograde traffic towards the soma, motor neurons were grown in microfluidic chambers, as described in Ch'ng, T. H. and Enquist, L. W. (2005) J Virol, 79, 10875-89; incorporated herein by reference. These silicone inserts allow culture of motor neurons so that they project axons through channels to a second chamber, allowing separation of the soma, axon, and axon tip. Neurons were purified as above and plated at 200,000 cells per dish into prepared and coated microfluidic chambers. 7 days post plating, an excess of viral particles (˜10,000) were added to the axonal chamber with or without Alexa-647-labelled anti-p75NTR as described in Restani, et al Traffic, 13, 1083-9; incorporated herein by reference, and incubated for 20 minutes at 37° C. before the chamber was filled with Neurobasal media, and left for a further 40 min, prior to imaging in an environmental chamber. For fixed imaging, vector particles were added to the axonal chamber in 8 μl for 20 min, before the chambers were filled with complete Neurobasal media and left for a total of 2 h to allow retrograde trafficking to occur. Neurons were fixed in 4% PFA, and then stained and processed as above. For transduction of motor neurons in microfluidic chambers, EGFP expressing vector particles were added to the axonal chamber in 8 μl for 20 min, before chambers were filled with complete neurobasal media and left for a total of 6 h to allow retrograde trafficking and transduction to occur. Medium was then replaced. 72 h post transduction, neurons were fixed in 4% PFA and stained with antibodies against EGFP (1:1000 ab290, Abcam), ChAT (1:100 AB144P, Millipore) and SM132 (1:1000 Covance, E11 CF00693).

To examine if these vectors undergo trafficking in certain reported pathways, lipid labeled (DiO) targeted vectors and controls (MOI 10) and also Alexa-647-labelled-p75NTR and Alexa-555-TeNT (tetanus neurotoxin) were applied to the axonal chamber. Labelling of the envelope with lipophilic dyes allowed highly efficient labeling of viral vectors without affecting the biological titer of the vectors.

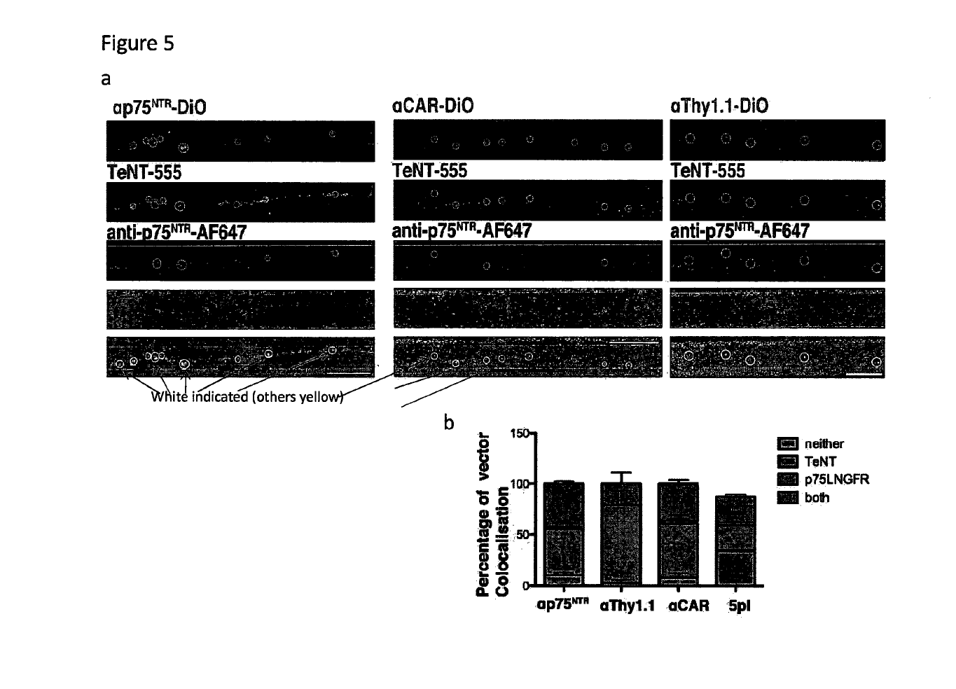

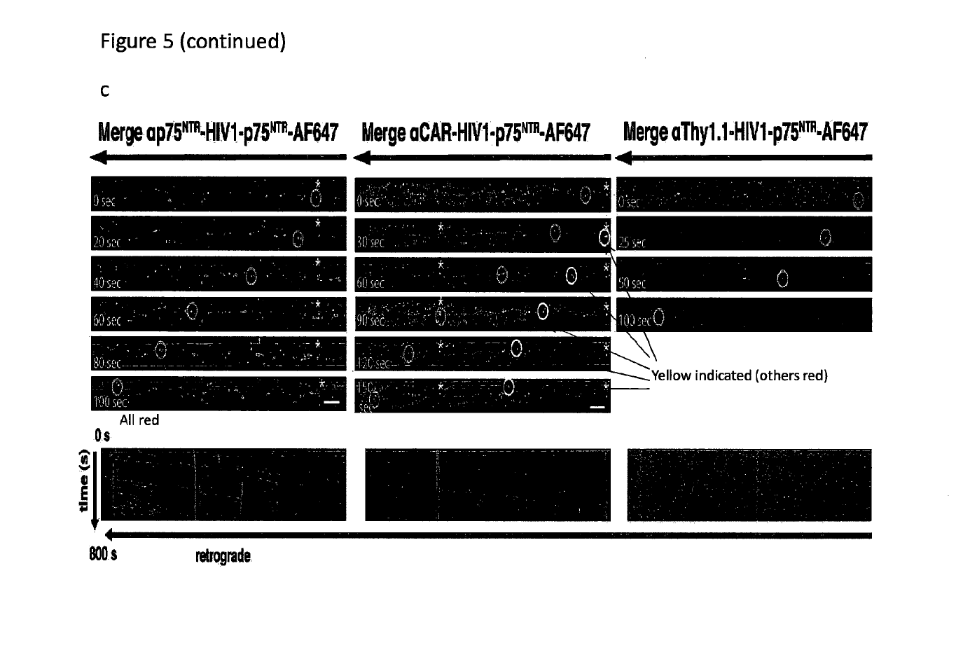

Fixed imaging of particles allowed extensive quantification of particles undergoing trafficking and of the endosomal pathway utilized. For αp75NTR-targeted vector, approximately 87% of the trafficked particles colocalised with the tetanus neurotoxin and the majority of these also colocalised with the p75NTR (more than 60%) (FIG. 5A). It has been reported that p75NTR internalizes upon activation and undergoes retrograde trafficking, in neuronal cells, in specialized, non-acidic endosomal compartments. Similar trafficking results were also observed for αCAR-targeted vector (FIG. 5A). In the case of the αThy1.1-targeted vector, a higher number of particles were predominantly colocalizing with the tetanus neurotoxin (approximately 93% of which 30% were also colocalizing with p75NTR). This further supports the specificity of αThy1.1 vector.

Importantly, these results were reproducible in all replications of the trafficking studies, and data obtained from analysis of at least 90 trafficking events per replication are shown. For all three vectors, a small number of particles not colocalizing with either marker were observed. This was more frequently observed for the non-targeted control vector. Using the same conditions, we were able to define the percentage of background reported from non-targeted control. Interestingly, non-targeted vector particles along the chambers were observed. Careful examination of these events revealed that the majority of these particles were not only non-associated with either marker but also detected outside the axon of the neuron. A small percentage of the particles observed were associated with one or both of the markers. It is noteworthy to highlight that we have been able to observe a maximum of 10-20 events in total in the same number of axons assessed, for trafficking as for the targeted vectors, suggesting that these events were rare and considered as background signal. Retrograde trafficking of targeted vectors was further confirmed when we applied HIV1 vector particles pseudotyped with VSVG to the same cultures (data not shown).

We next attempted to determine retrograde trafficking of these vectors in live primary MNs. Live imaging was performed as described in Salinas et al PLoS Pathog, 5, e1000442; incorporated herein by reference. Briefly, cells were incubated with labelled LV particles (MOI 10) for 15 min, before cells were washed once and imaging media added. Dishes were then imaged using a TCS SP5 II confocal microscope with environmental chamber. Images were taken every 5 sec using the 488 nm laser, with the photo multiplier tube detection range of 500-550 nm, as DiO fluoresces in the green spectrum.

Images were processed using ImageJ and images enhanced using a 2.0 pixel median filter and brightness/contrast, and particle analysis performed using MTrackJ and multiple Kymograph plugins.

Time-lapse confocal microscopy following co-application of either of the targeted vectors and anti-p75NTR for 60 min showed that retrograde transport for all three targeted vectors was easily detectable (FIG. 5B). Most of the vector trafficking occurred within the same endosomal compartment as the p75NTR. We observed vectors colocalised with p75NTR but not undergoing trafficking or vector non-associated with p75NTR but undergoing trafficking. These events were rare compared to the majority of the moving particles, which appeared to be associated with p75NTR at rates similar to those we have observed in fixed cultures. Particle analysis of these vectors demonstrated that they were potent to undergo extensive trafficking (>100 μm). Speed and number of events varied among the three vectors, with αCAR and αp75NTR vectors travelling along the axons with similar efficiency (FIG. 5B).

Taken together, these results reveal that all three targeted vectors are readily able to undergo retrograde endosomal transport, upon application to the axon tip of the target motor neuron. Interestingly, all three vectors appeared to have better trafficking characteristics than the ones observed for RVG pseudotyped lentiviral vectors (data not shown). Number of particles observed to traffic along the same number of axons assessed, were in average 42 events for each repetition for RVG preparations of similar titer to any of the targeted ones, where in average 90 events were observed for each repetition. Endocytosed cargo undergoes a journey along the axons, though vector fusion with the endosome and escaping into the cytosol is dependent on the envelope used in this study, which is the mutated version of Sindbis virus for efficient endosomal escape.

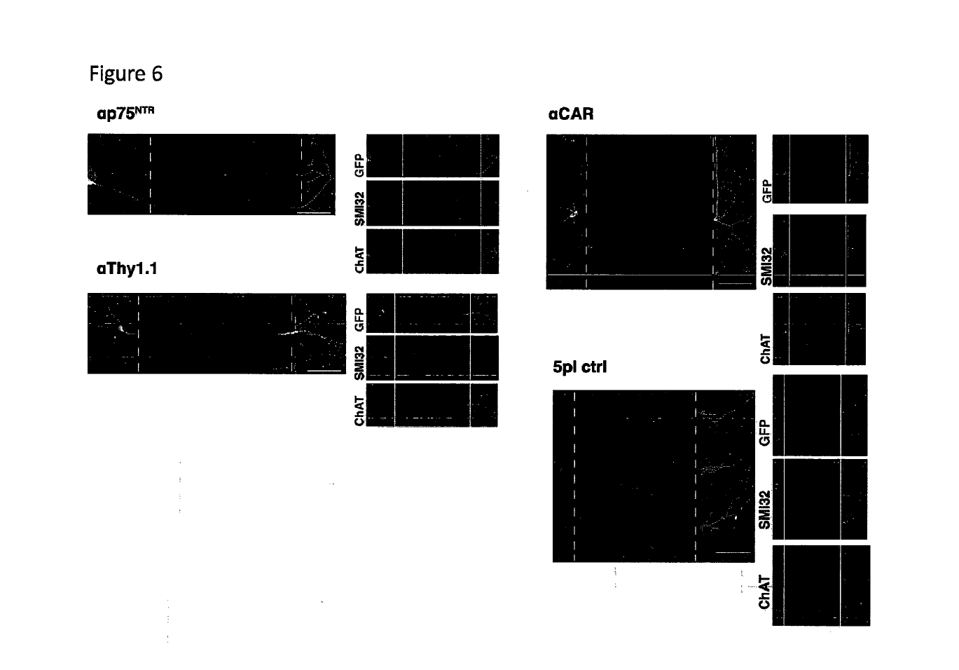

Since we confirmed that particles move along the axon, our next question was, whether they reach the soma and if so, whether endosomal escape occurs on time to achieve transduction of the targeted neuron. To assess this we applied to the axonal side EGFP-expressing targeted vectors and controls and assessed transduction though EGFP expression via immunostaining and confocal microscopy. Examples of transduced motor neuron axons, crossing the microgroove of the MCF and transduced soma, positive for EGFP, SM132 (axonal marker) and ChAT (choline acetyltransferase) are shown in FIG. 6. Transduced motor neurons were observed for all three targeted vectors. Axons belonging to a different type of neuron (or a motor neuron not being transduced) not expressing EGFP are visible in adjacent parts of the panels. The analysis of these data was only qualitative. Determination of MOI used was difficult, since at DIV7 it was impossible to determine not only the number of axons grown along the microgrooves to which the vectors were initially applied to, but also the somas of the neuron from which they have originated. Transduction was not observed for either 5pI control or VSVG-pseudotyped HIV-1 vector particles. Transduction experiments performed with wild-type RVG pseudotyped HIV-1 vector, where the same number of particles was applied to the axonal compartment, resulted in no or extremely poor transduction, at background levels (data not shown). Taken together these results indicate that these newly engineered targeted vectors are more efficient than RVG pseudotyped HIV1 in trafficking and transduction of primary MNs, in vitro.

### Example 5

### The Lentiviral Vectors Permit Targeted In Vivo Gene Transfer

It was investigated whether the lentiviral vectors are capable of delivery directly to spinal cord MNs via the NMJ. It has been shown that transduction of spinal cord motor neurons with rabies virus glycoprotein (RVG) pseudotyped lentiviral vectors can be achieved upon peripheral administration to gastrocnemious muscle (Mazarakis, N. D., et al (2001) Hum Mol Genet, 10, 2109-21; and Mentis, G. Z et al (2006) J Neurosci Methods, 157, 208-17; both of which are incorporated herein by reference).

In this case, small incisions in hind limb were performed to expose tibia/is anterior (TA) muscle. Concentrated targeted lentiviral αCAR (25 μl of 1.0×10⁹ TU/ml) EGFP expressing vector and 5pI control (25 μl of 3.6×10⁹ TU/ml) were intramuscularly injected unilaterally in right tibialis anterior muscle of C57BL/6 mice (n=3) via single injection with a 26G hypodermic needle. Three weeks post injection mice were euthanized by intraperitoneal injection of an 200 mg/kg of sodium pentobarbitone, transcardially perfused with 10 ml saline (0.9% w/v NaCl) plus heparin, followed by 100 ml of 4% (wt/vol) paraformaldehyde (PFA, Sigma Aldrich) in PBS. Spinal cord and tibialis anterior muscles were removed and post-fixed for 4 hours in 4% PFA, followed by cryoprotection in 10% glycerol and 20% sucrose in PBS for at least 72 h. Tissues were subsequently embedded and frozen in OCT (Surgipath FSC22, Leica Microsystems). 50 μm coronal sections from sacral, through the entire lumbar part of spinal cord were cut using a cryostat (Leica Microsystems, Wetzlar, Germany), mounted on gelatinized slides and stored at −20° C.

Immunohistochemistry was performed on slide mounted spinal cord sections. Tissue was permeabilised for 1 hour with PBS containing 10% donkey serum and 0.1% Triton-X100. Antibodies to EGFP (1:500, ab290, Abeam), ChAT (1:50 AB114P, Millipore) were diluted in the same buffer and placed on sections for 72 h at 4° C. Sections were then blocked for 30 min prior to incubating for 3 h at room temperature with donkey anti-rabbit, anti-goat secondary antibodies conjugated with AlexaFluor® 488 or 594 respectively (1:400) (Invitrogen, Paisley, UK). Sections were mounted with ProLong anti-fade aqueous mounting medium (Invitrogen, Paisley, UK).

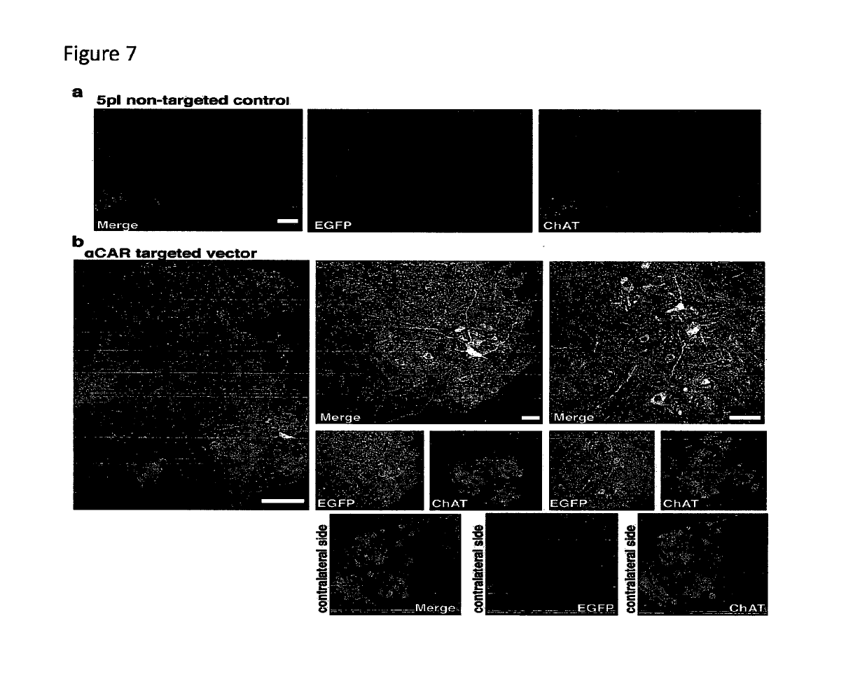

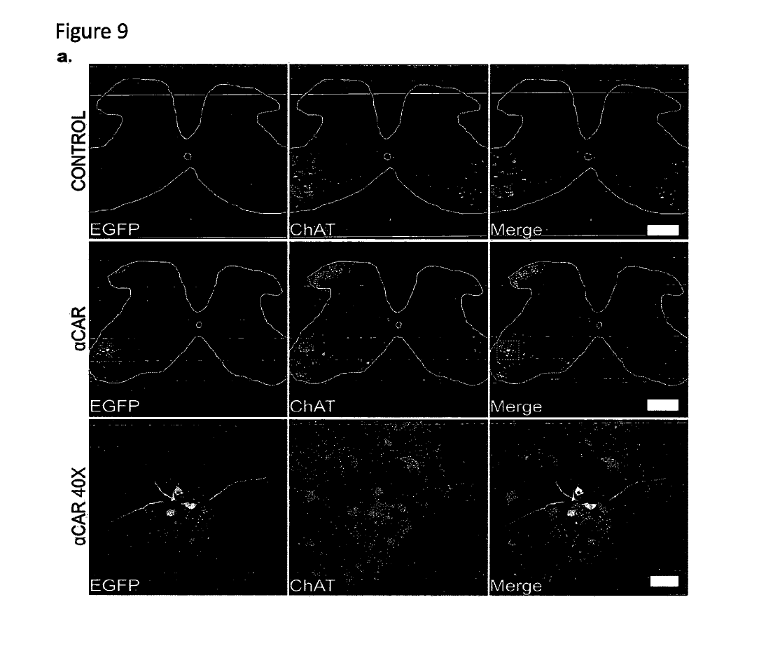

After 3 weeks, injections with control did not result in any detectable transduction of spinal cord cells (FIGS. 7 and 9). In contrast, EGFP transgene expression was observed in neurons within the lumbar cord region of the spinal cord of mice injected with αCAR-targeted vector (FIGS. 7 and 9). The insert of merge image (FIG. 9) shows a panel of five EGFP-expressing motor neuron cells. Both the soma and motor neuron dendrites were labeled with variable intensities (FIG. 9). To identify these EGFP-positive cells as motor neurons, immunostaining was performed for ChAT, which allowed us to follow transgene expression in these motor neurons for a series of 5-6 sections (50 μm each). Viral transduction was also observed with both vectors at the injection site (muscle fibers, data not shown). Despite the successful transduction of spinal motor neuron cells as a result of intramuscular injection of the αCAR-targeted vector, we did observe variability of transduction between animals.

To examine and determine whether transport to the CNS form peripheral muscle injection sites (TA) could be visualized prior to 3 weeks, concentrated targeted lentiviral αCAR (25 μl of 1.9×109 TU/ml) Luciferase (firefly luciferase) expressing vector was injected unilaterally in C57BL/6 right Tibialis Anterior muscle. 5pI non-targeted control (25 μl of 1.4×109 TU/ml) was similarly injected. Prior to imaging, mice were injected intraperitoneally with 150 mg of D-luciferin (Gold Biotechnology, St. Louis, USA). The reporter gene (firefly luciferace) expression was imaged at days 7, 14 and 21 post injection using a bioluminescence imaging system (IVIS Spectrum/200, Perkin Elmer). At final time point (21 days) animals were subjected to terminal anesthesia. Images were analysed by using Living Image 4.3.1 software.

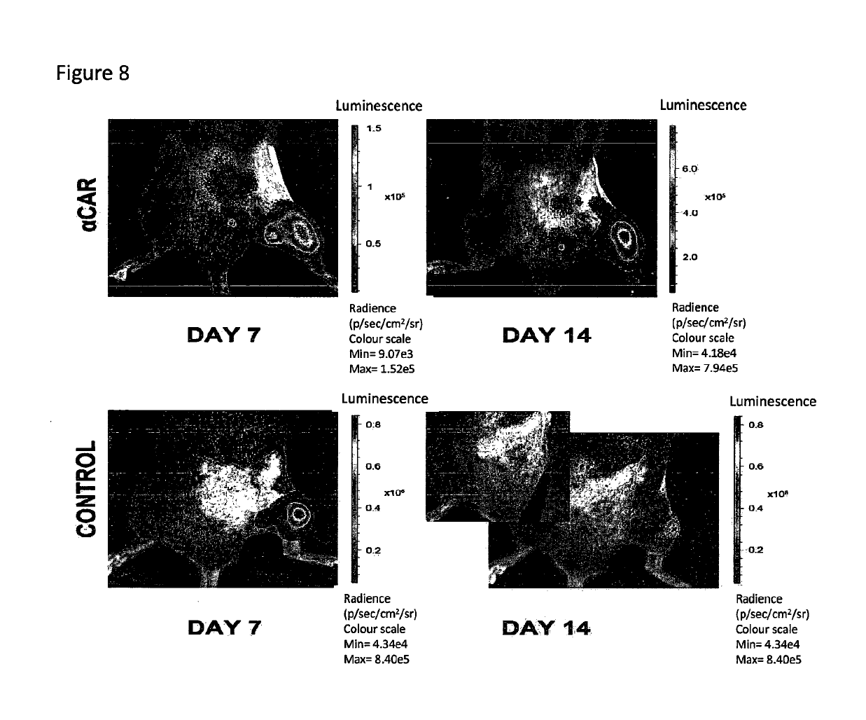

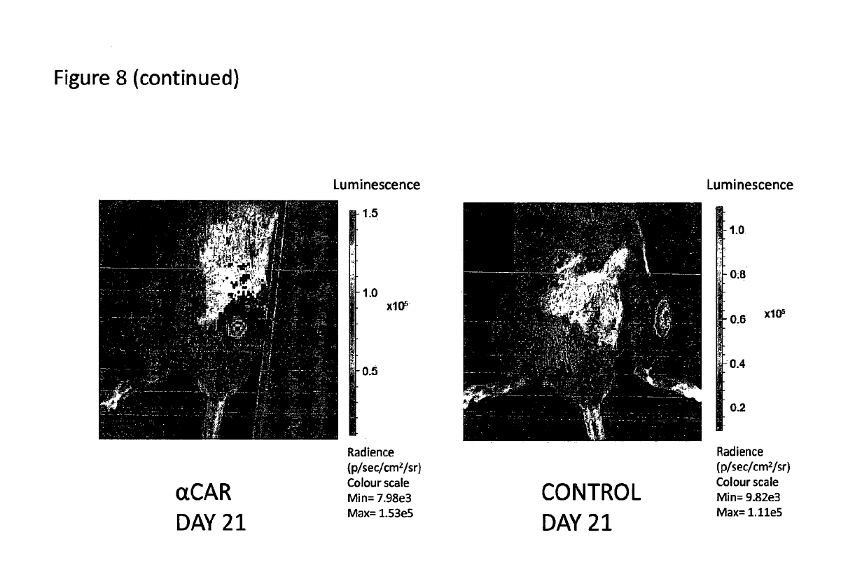

The expression of the delivered reporter gene was monitored and results are shown in FIG. 8. For the mice injected with the αCAR-targeted vector, two strong bioluminescent signals generated by firefly luciferase expression were seen at the injection site and at the level of the spinal cord. In contrast, administration of non-targeting 5pI control generated bioluminescence signals only specific to the muscle.

These results demonstrate validation of the route in vivo, and allow us to observe transport and expression of transgene dynamically upon muscle application. The vectors we developed here can be particularly useful for minimally invasive administration of the vectors by targeting the peripheral sites of the neuromuscular synapse in order to ultimately reach the affected CNS. We have based our approach on incorporating membrane bound antibodies against presynaptic terminal receptors Thy1.1, p75NTR and CAR on the surface of lentiviral vectors pseudotyped with modified Sindbis GP.

Overall, the results presented in this study indicate that these newly engineered targeted vectors bear novel trafficking and transduction characteristics and specificity for MNs, superior to ones observed with existing lentiviral vectors, which make them good candidates for non-invasive CNS therapy for ALS.

1. A lentiviral vector comprising (i) an antibody that binds to a pre-synaptic terminal receptor on the neuromuscular junction (NMJ); and (ii) a fusogenic molecule.

2. The lentiviral vector of claim 1, wherein the antibody binds to a receptor or binding site on the NMJ which is capable of endocytosis.

3. The lentiviral vector of claim 1, wherein the antibody is an antibody that binds to a Thy-1 receptor, a CAR (coxsackievirus and adenovirus receptor) and/or a p75 (low-affinity neurotrophin receptor).

4. The lentiviral vector of claim 1, wherein the fusogenic molecule is a glycoprotein.

5. The lentiviral vector of claim 4, wherein the glycoprotein is a viral glycoprotein.

6. The lentiviral vector of claim 5, wherein the viral glycoprotein is a mutated Sindbis virus glycoprotein that is binding defective and fusion competent.

7. The lentiviral vector claim 1, wherein the fusogenic molecule is a class I or class II fusogen.

8. The lentiviral vector of claim 1, further comprising a therapeutic agent.

9. The lentiviral vector of claim 8, wherein the therapeutic agent is a CNS-targeted therapeutic agent; a neuroprotective protein; insulin-like growth factor; heat shock protein 70 (HSP-70); or a neurotrophic factor.

10. A method of treating a neural disorder, the method comprising administering a lentiviral vector of claim 1.

11. (canceled)

12. The method of claim 10, wherein the neural disorder is amyotrophic lateral sclerosis (ALS); primary lateral sclerosis; progressive muscular atrophy; spinal muscular atrophy; progressive bulbar palsy; or pseudobulbar palsy.

13. The lentiviral vector of claim 1, wherein the fusogenic molecule is a hemagglutinin.

14. The lentiviral vector of claim 5, wherein the viral glycoprotein is selected from a Sindbis virus glycoprotein, a Lassa fever virus glycoprotein, a tick-borne encephalitis virus glycoprotein, a dengue virus glycoprotein, a hepatitis B virus glycoprotein, a rabies virus glycoprotein, a Semliki Forest virus glycoprotein, a Ross River virus glycoprotein, an Aura virus glycoprotein, a Borna disease virus glycoprotein, a Hantaan virus glycoprotein, or a SARS-CoV virus glycoprotein.

15. The lentiviral vector of claim 6, wherein the mutated Sindbis virus glycoprotein is SINmu or SINmu(SGN).

16. The lentiviral vector of claim 8, wherein the therapeutic agent is a RNAi or siRNA targeting a neural disorder-associated dominant negative mutation.

17. The lentiviral vector of claim 8, wherein the therapeutic agent is gene therapy to restore a wild-type gene that is inactive in a neural disorder.

18. The lentiviral vector of claim 17, wherein the neural disorder is spinal muscular atrophy and the wild-type gene being restored is a survival motor neuron 1 (SMN1) gene.

19. The method of claim 10, wherein the fusogenic molecule is a viral glycoprotein selected from a Sindbis virus glycoprotein, a Lassa fever virus glycoprotein, a tick-borne encephalitis virus glycoprotein, a dengue virus glycoprotein, a hepatitis B virus glycoprotein, a rabies virus glycoprotein, a Semliki Forest virus glycoprotein, a Ross River virus glycoprotein, an Aura virus glycoprotein, a Borna disease virus glycoprotein, a Hantaan virus glycoprotein, or a SARS-CoV virus glycoprotein.

20. The method of claim 19, wherein the viral glycoprotein is a Sindbis virus glycoprotein, the Sindbis virus glycoprotein mutated to be binding defective and fusion competent.

21. The method of claim 10, further comprising administering a neural disorder therapeutic agent.
